(19) United States
(12) Patent Application Publication
(10) Pub. No.: US 2016/0168538 A1
(43) Pub. Date: Jun. 16, 2016

(54) Flk1+ and VE-Cadherin+ Endothelial Cells Derived from iPS or ES Cells, and Methods of Preparing and Using the Same

(71) Applicant: THE BOARD OF TRUSTEES OF THE UNIVERSITY OF ILLINOIS, URBANA, IL (US)

(72) Inventors: Kishore K. Wary, Elmhurst, IL (US); Asrar Malik, Hinsdale, IL (US); Jalees Rehman, Willowbrook, IL (US)

(73) Assignee: THE BOARD OF TRUSTEES OF THE UNIVERSITY OF ILLINOIS, URBANA, IL (US)

(21) Appl. No.: 14/969,462

(22) Filed: Dec. 15, 2015

Related U.S. Application Data
(60) Provisional application No. 62/091,757, filed on Dec. 15, 2014.

(51) Int. Cl. C12N 5/071; A61K 35/44

(52) U.S. Cl. CPC C12N 5/069; A61K 35/44; C12N 2506/45; C12N 2501/998; C12N 2501/155; C12N 2501/165; C12N 2501/115

(57) ABSTRACT

This invention provides endothelial cells expressing transcription factor Er71/Etv2, and cell surface endothelial markers Flk1 and VE-cadherin prepared from induced pluripotent stem cells or embryonic stem cells, as well as methods of preparing the cells and methods of using the cells to vascularize and re-endothelialize or repair ischemic tissue in a subject.

### INTRODUCTION

This application claims the benefit of priority of U.S. Provisional Application No. 62/091,757, filed Dec. 15, 2014, the content of which is incorporated herein by reference in its entirety.

This invention was made with government support under contract numbers 5R01HL090152, 2 R01HL079356 and 5R01GM094220 awarded by the National Institutes of Health. The government has certain rights in the invention.

### BACKGROUND

The transduction of fibroblast cells with transcription factors Nanog, Sox2(SRY-related HMG-box 2), Oct4(Octamer-binding transcription factor 4), Klf4 (Kruppel-Like Factor 4), and c-Myc (V-myc avian myelocytomatosis viral oncogene homolog) converts these cells into induced pluripotent stem (iPS) cells. The observations that adult mice can be derived from iPS cells indicate that these reprogrammed cells acquire embryonic stem (ES) cell-like properties, and therefore have the potential to generate any tissue (Boland, et al. (2009) Nature 461:91-94; Quinlan, et al. (2011) Cell Stem Cell 9:366-373). An important aim of regenerative cell therapy is to use the iPS cells because they not only self-renew and have the potential to differentiate into mature cells. However, unlike ES cells, iPS cells can give rise to autologous cells that are ideal for personalized regenerative therapies.

During embryogenesis, primitive vascular endothelial cells (ECs), termed angioblasts, and hematopoietic stem cells emerge from the mesodermal compartment in successive waves to form blood vessels. The upstream components that induce exit of mesodermal cells to vascular cell progenies include factors such as bone morphogenetic proteins (BMPs), hypoxia, and Wnts. A subset of mesodermal cells expressing Flk1⁺Flt1⁺VE-cadherin⁺CD34⁺CD31⁺ are capable of forming vascular plexus-like structures. Fetal liver kinase (Flk)-1 (in mice), referred to as vascular endothelial growth factor receptor (VEGFR)-2(in humans), acts as an earliest marker of mesodermal stem cells and angioblasts. In mice, Flk1⁺ cells differentiate into ECs to form primitive vascular structures through the process of vasculogenesis. Binding to vascular endothelial growth factor (VEGF) to Flk1/VEGFR-2 regulates multiple aspects of neovascularization including EC development, survival, differentiation, migration, and lumenization. The one-pass transmembrane protein VE-cadherin, which mediates cell-cell adhesion and contributes to the formation of adherens junctions (AJs), is expressed in both immature and mature ECs. Analysis of the endothelial promoter/enhancer revealed the presence of ETS (E-twenty six) binding site that directly regulate expression of most, if not all, endothelial genes. The transcription factors Er71(Ets-related protein 71, also known as Etv2/Etsrp), FoxC2 (Forkhead box protein C2), ERG1(ETS-related gene-1) and Fli1(Friend leukemia integration 1 transcription factor) were shown to regulate the development of vascular ECs. Thus, the development of ECs entails timely expression and function of above key proteins.

In adults, there is only a limited pool of endothelial progenitor cells (EPCs) that contribute to neovascularization and repair in situ, and these EPCs are often dysfunctional or lost in patients with cardiovascular risk factors. Methods for using pluripotent stem cells to produce endothelial cells or regenerative cells have been described. See, e.g., U.S. Pat. No. 8,785,192, US 2012/0301443, US 2012/0315257; WO 2012/168167, WO 2013/130820 and WO 2013/166165. Further, directional differentiation of mouse iPS cells to cardiovascular cells has been demonstrated (Narazaki, et al. (2008) Circulation 118:498-506). Similarly, intravenously administered iPS cell-derived Flk-1⁺cells are shown to be recruited to a site of vascular injury and enhance reendothelialization followed by suppression of neointimal hyperplasia (Yamamoto, et al. (2013) Arterioscler. Thromb. Vasc. Biol. 33:2218-2221). However, it has not been determined whether iPS cells can be used as a source of reparative ECs to induce revascularization. It is also not known whether miPS and mouse embryonic stem (ES) cell-derived ECs have similar pattern of differentiation and function to induce vascularization.

### SUMMARY OF THE INVENTION

This invention is a method of preparing endothelial cells that express Ets-related protein 71(Er71, also known as Etv2/Etsrp), Fetal liver kinase-1(Flk1, also known as VEGFR2 and KDR) and Vascular Endothelial (VE)-cadherin by (i) culturing induced pluripotent stem cells or embryonic stem cells in the presence of Type IV collagen, Bone Morphogenetic Protein 4(BMP4), Vascular Endothelial Growth Factor (VEGF) and basic Fibroblast Growth Factor (bFGF); and (ii) isolating endothelial cells that express transcription factor Er71, and vascular endothelial cell surface markers Flk1 and VE-cadherin proteins. In one embodiment, the type IV collagen includes α1(IV) and α2(IV) chains. In another embodiment, the induced pluripotent stem cells or embryonic stem cells are cultured for about 1 to about 5 days. In some embodiments, the endothelial cells are isolated by fluorescence-activated cell sorting using anti-Flk1 and anti-VE-cadherin antibodies. In other embodiments, the endothelial cells express Flk1, VE-cadherin and CD31, but are negative for CD14, CD45, or a combination thereof. A substantially pure population of endothelial cells is also provided, as is a composition or bioengineered tissue graft containing the substantially pure population of endothelial cells. Use of the endothelial cells in a method of promoting vascularization or repair of injured or ischemic tissue, e.g., cardiac tissue, liver tissue, pancreatic tissue, renal tissue, muscle tissue, neural tissue, or bone tissue, is also encompassed by this invention.

### BRIEF DESCRIPTION OF THE DRAWINGS

FIG. 1 shows the timeline of emergence of Flk1⁺VE-cadherin⁺ vascular ECs.

FIGS. 2A-2I show the gene expression profiles of ECs derived from mES and miPS cells. Total mRNAs were prepared from mES and miPS cells at day 0 and FACS-sorted Flk1⁺VE-cadherin⁺ ECs at day 5, and subjected to q-RT-PCR analysis thereafter. FIGS. 2A-2D show the expression of transcription factors c-Myc (FIG. 2A), Oct 4(FIG. 2B), Klf4(FIG. 2C), and Sox2(FIG. 2D) in mES cells (ESC) and miPS cells (iPSC) on the indicated days. Decreased expression of Brachyury, but increased Er71 indicated development of mesodermal-endothelial progenies (FIGS. 2E and 2F). Highly enriched expression of Flk1, VE-cadherin, and CD31 transcripts indicated acquisition of the EC phenotype (FIGS. 2G-2I). Data represent mean±S.E.M., n=5. Experiments were repeated at least 5 times with quadruplicates.

FIG. 3 shows schematics of promoter/enhancer regions of Flk1- (−1.4 kb), VE-cadherin- (−1.0 kb), and CD31- (−1.0 kb) harboring GGAA sites (triangles represent GGAA sites on the forward strand and asterisks represent these in the reverse strand), and the number within parentheses indicates the number of putative Er71 binding sites; TSS, transcription start site.

FIG. 4 shows the angiogenic potential of Flk1⁺VE-cadherin⁺ ECs derived from mES and iPS cells. Control mouse ECs (EC) and mES-derived (ES) or iPS-derived (iPS) Flk1⁺ VE-cadherin⁺ ECs (2×10⁵) were plated onto MATRIGEL in the presence of VEGF¹⁶⁵ (50 ng/ml). After 18 hours, the numbers of branching points were counted. Data are expressed as percentage of branching points (n=3, *P<0.05 vs. control or as indicated).

FIGS. 5A and 5B show that mouse iPS-derived Flk1⁺ VE-cadherin⁺ cells incorporate into CD31-positive vessels. FIG. 5A shows the time line of the MATRIGEL experiment. FIG. 5B shows the quantification of number of neovessels in MATRIGEL sections incorporating the control mouse ECs (EC) and mES-derived (ES) or iPS-derived (iPS) Flk1⁺VE-cadherin⁺ cells expressing mCherry. Scale bar 100 μm. Experiments were repeated at least 3 times.

### DETAILED DESCRIPTION OF THE INVENTION

It has now been demonstrated that naïve embryonic stem (ES) cells and induced pluripotent stem (iPS) cells cultured in type IV collagen (IV Col) in defined media for about 1-5 days induces the formation of adherent cell populations, which express of transcription factor Er71, and vascular endothelial cell surface markers Flk1(Fetal liver kinase-1; also known as KDR or VEGFR-2) and VE (Vascular Endothelial)-cadherin (CD144) and exhibit emergence of endothelial cell (EC) progenies. FACS purification results in 100% Flk1⁺ VE-cadherin⁺ cells from both ES and iPS cells. Emergence of Flk1⁺VE-cadherin⁺ cells entails expression of the vascular developmental transcription factor Er71, which binds identically to Flk1, VE-cadherin, and CD31 promoters in both populations. Immunostaining with anti-VE-cadherin and anti-CD31 antibodies and microscopy demonstrates the endothelial nature of these cells. Each cell population (unlike mature ECs) organizes into well-developed vascular structures in vitro and incorporates into CD31⁺ neovessels in MATRIGEL plugs implanted in nude mice in vivo. Thus, iPS cell-derived Flk1⁺VE-cadherin⁺ cells expressing the developmental transcription factor Er71 are as angiogenic as ES cell-derived cells and incorporate into CD31⁺ neovessels. Their vessel forming capacity highlights the potential of autologous ES-and iPS cell-derived EC progeny for therapeutic angiogenesis. Accordingly, the present invention provides a method of preparing endothelial cells that co-express Er71, Flk1 and VE-cadherin proteins as well as isolated endothelial cell populations and methods of using the same in cell therapy applications.

In accordance with this invention, endothelial cells co-expressing Er71, Flk1 and VE-cadherin are prepared by (i) culturing iPS cells or ES cells in the presence of type IV collagen, BMP4 (Bone Morphogenetic Protein 4), VEGF (Vascular Endothelial Growth Factor) and bFGF (basic Fibroblast Growth Factor); and (ii) isolating the cells co-expressing Er71, Flk1 and VE-cadherin. In some embodiments, the factors for differentiating iPS cells or ES cells into endothelial cells consist of or consist essentially of type IV collagen, BMP4, VEGF and bFGF.

As is known in the art, “embryonic stem cells,” commonly abbreviated as ES cells or ESCs, are pluripotent stem cells derived from early embryos. “Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by inserting certain genes, referred to as reprogramming factors. Embryonic stem cells and induced pluripotent stem cells are capable of long-term proliferation in vitro, while retaining the potential to differentiate into all cell types of the body, including endothelial cells. Thus, these cells could potentially provide an unlimited supply of patient-specific functional endothelial cells for both drug development and therapeutic uses. In addition, human ESC/iPSCs, with their unlimited proliferation ability, have a unique advantage over somatic cells as the starting cell population for endothelial cell differentiation.

Embryonic Stem Cells. Embryonic stem cell lines (ES cell lines) are cultures of cells derived from the epiblast tissue of the inner cell mass (ICM) of a blastocyst or earlier morula stage embryos. A blastocyst is an early stage embryo that is approximately four to five days old in humans and is composed of 50-150 cells. ES cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

A significant amount of research has taken place using mouse embryonic stem cells or human embryonic stem cells. Both have the essential stem cell characteristics and can be maintained in an undifferentiated state. Mouse ES cells may be grown on a layer of gelatin and require the presence of Leukemia Inhibitory Factor (LIF). Human ES cells could be grown on a feeder layer of mouse embryonic fibroblasts (MEFs) and often require the presence of basic Fibroblast Growth Factor (bFGF or FGF-2). Without optimal culture conditions or genetic manipulation (Chambers, et al. (2003) Cell 113(5):643-55), embryonic stem cells will rapidly differentiate.

Methods for obtaining mouse ES cells are well-known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith (2000) In: Origins and Properties of Mouse Embryonic Stem Cells, Annu. Rev. Cell. Dev. Biol.). In other methods, mouse ES cells can be grown in serum-free medium in the presence of bone morphogenetic protein and LIF (Ying, et al (2003) Cell 115:281-292).

Human ES cells can be obtained from blastocysts using established described methods (Thomson, et al. (1995) Proc. Natl. Acad. Sci. USA 92:7844-7848; Thomson, et al. (1998) Science 282:1145; Thomson & Marshall (1998) Curr. Top. Dev. Biol. 38:133-165; Reubinoff, et al. (2000) Nat. Biotechnol. 18:399-404) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e., exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit, et al. (2000) Dev. Biol. 227:271-278). In other methods, human ES cells can be grown without a feeder cell layer by culturing the cells on a protein matrix such as MATRIGEL or laminin in the presence of “conditioned” medium containing basic fibroblast growth factor (Xu, et al. (2001) Nat. Biotechnol. 19:971-974). The medium is previously conditioned by co-culturing with fibroblasts.

Methods for the isolation of rhesus monkey and common marmoset ES cells are also known (Thomson & Marshall (1998) Curr. Top. Dev. Biol. 38:133-165; Thomson, et al. (1995) Proc. Natl. Acad. Sci. USA 92:7844-7848; Thomson & Odorico (2000) J. Trends. Biotechnol. 18:53-57).

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines HI, H7, H9, H13 and H14 are known and established cell lines. In addition, subclones H9.1 and H9.2 of the H9 line have been developed. Moreover, undifferentiated mouse embryonic stem cells, such as the J1 line, are available from commercial sources such as American Type Culture Collection (ATCC, Manassas, Va.). It is anticipated that virtually any ES or stem cell line known in the art may be used with the present invention, such as, e.g., those described in Yu & Thompson (2008) Genes Dev. 22(15):1987-97.

The source of ES cells for use in connection with the present invention can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

Human embryonic stem cells may be defined by the presence of several transcription factors and cell surface proteins. The transcription factors Oct-4, Nanog, and Sox-2 form the core regulatory network that ensures the suppression of genes that lead to differentiation and the maintenance of pluripotency (Boyer, et al. (2005) Cell 122(6):947-56). The cell surface antigens most commonly used to identify hES cells include the glycolipids SSEA3 and SSEA4 and the keratan sulfate antigens Tra-1-60 and Tra-1-81.

Induced Pluripotent Stem Cells. iPS cells are cells that have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi, et al. (2007) Cell 126(4):663-76). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF. Based on cell characteristics, cells of the ES cell-like colonies are induced pluripotent stem cells. The induced pluripotent stem cells are morphologically similar to human ES cells, and express various human ES cell markers. Also, when grown under conditions that are known to result in differentiation of human ES cells, the induced pluripotent stem cells differentiate accordingly. For example, as demonstrated herein, the induced pluripotent stem cells can differentiate into cells having endothelial cell structures and endothelial cell markers. It is anticipated that virtually any iPS cells or cell lines may be used with the present invention, including, e.g., those described in Yu & Thompson (2008) Genes Dev. 22(15):1987-97.

In another method, human fetal or newborn fibroblasts are transfected with four genes, Oct4, Sox2, Nanog and Lin28 using lentivirus transduction (Yu, et al. (2007) Science 318:1917-1920). At 12-20 days post-infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage, and gut epithelium after injection into mice.

Methods of preparing induced pluripotent stem cells from mice are also known (Takahashi & Yamanaka (2006) Cell 126:663-676). Induction of iPS cells typically requires the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc. Established mouse iPS cells include, e.g., iMZ-9 and iMZ-21(Boland, et al. (2009) Nature 461:91-94; Quinlan, et al. (2011) Cell Stem Cell 9:366-373)

iPS cells, like ES cells, have characteristic antigens that can be identified or confirmed by immunohistochemistry or flow cytometry using antibodies for SSEA-1, SSEA-3, SSEA-4, TRA-1-60 and TRA-1-81(Andrews, et al. (1987) In: Teratocarcinomas and Embryonic Stem Cells, Robertson (Ed.), IRL Press, 207-246). Pluripotency of embryonic stem cells can be confirmed by injecting approximately 0.5-10×10⁶ cells into the rear leg muscles of 8-12 week old male SCID mice. Teratomas develop that demonstrate at least one cell type of each of the three germ layers.

In certain aspects of the present invention, iPS cells are made from reprogramming somatic cells using reprogramming factors including an Oct family member and a Sox family member, such as Oct4 and Sox2 in combination with Klf or Nanog. The somatic cell for reprogramming may be any somatic cell that can be induced to pluripotency, such as a fibroblast, a keratinocyte, a hematopoietic cell, a mesenchymal cell, a liver cell, a stomach cell, or a β cell. In particular embodiments, the iPS cells are prepared by transducing embryonic fibroblasts with lentiviruses containing Oct4, Klf4, Sox2 and c-Myc. See, e.g., Boland, et al. (2009) Nature 461:91-94; Quinlan, et al. (2011) Cell Stem Cell 9:366-373.

Reprogramming factors may be expressed from expression cassettes included within one or more vectors, such as an integrating vector or an episomal vector, e.g., an EBV element-based system (see, e.g., Yu, et al. (2009) Science 324(5928):797-801). In a further aspect, reprogramming proteins could be introduced directly into somatic cells by protein transduction.

In accordance with the method of this invention, iPS or ES cells are cultured on type IV collagen in the presence of BMP4, VEGF and bFGF, or isoforms or variants thereof, to induce differentiation of the iPS or EC cells into endothelial cells expressing Flk1 and VE-cadherin.

Type IV collagen has been shown to be a major structural component of basement membranes. The protomeric form of type IV collagen is formed as a heterotrimer made up from a number of different subunit chains called α1(IV) through α6(IV). Six genetically distinct a-chains belonging to two classes with extensive homology have been identified, and their relative abundance has been demonstrated to be tissue specific. The type IV collagen heterotrimer is characterized by three distinct structural domains: the non-collagenous (NC1) domain at the carboxyl terminus; the triple helical collagenous domain in the middle region; and the 7S collagenous domain at the amino terminus. (Martin, et al. (1988) Adv. Protein Chem. 39:1-50; Gunwar, et al. (1991) J. Biol. Chem. 266:14088-14094). In certain embodiments of this invention, the type IV collagen used comprises or consists of α1(IV) and α2(IV) chains.

Bone Morphogenetic Protein 4(BMP4) is important for the modulation of the proliferative and differentiative potential of hematopoietic progenitor cells (Bhardwaj, et al. (2001) Nat. Immunol. 2:172-180; Bhatia, et al. (1999) J. Exp. Med. 189:1139-1148; Chadwick (2003) Blood 102(3):906-91). Additionally, BMP4 can modulate early hematopoietic cell development in human fetal, neonatal, and adult hematopoietic progenitor cells (Davidson & Zon (2000) Curr. Top Dev. Biol. 50:45-60; Huber, et al. (1998) Blood 92: 4128-4137; Marshall, et al. (2000) Blood 96:1591-1593). For example, BMP4 can regulate the proliferation and differentiation of highly purified primitive human hematopoietic cells from adult and neonatal sources (Bhatia, et al. (1999) J. Exp. Med. 189:1139-1148), and BMP4 can promote hematopoietic differentiation in human embryonic stem cells (Chadwick (2003) Blood 102(3):906-91). BMP4 can also promote differentiation of endothelial cells from endothelial progenitor cells (Wang, et al. (2007) Nature Biotech. 25(3):317-318).

Vascular Endothelial Growth Factor (VEGF) is an important signaling protein that is involved in formation of the embryonic circulatory system and angiogenesis. VEGF can affect a variety of cell types including vascular endothelium and other cell types (e.g., neurons, cancer cells, kidney epithelial cells). In vitro, VEGF can stimulate endothelial cell mitogenesis and cell migration. VEGF function has also been shown to be important in a variety of disease states including cancer, diabetes, autoimmune diseases, and ocular vascular diseases.

Basic Fibroblast Growth Factor (bFGF) is present in basement membranes and in the subendothelial extracellular matrix of blood vessels. bFGF is a known component of human embryonic stem cell culture medium and mouse-feeder cell dependent culture systems, as well as in feeder and serum-free culture systems (Liu, et al. (2006) Biochem. Biophys. Res. Commun. 346:131-9. bFGF, in conjunction with BMP4, promote differentiation of stem cells to mesodermal lineages.

The base medium for culturing and inducing differentiation of the iPS or ES cells can be any suitable endothelial cell differentiation medium, which is a nutrient-rich buffered solution capable of sustaining cell growth. Culture media suitable for differentiating iPS and ES cells into endothelial cells according to the method described herein include, but are not limited to, high glucose Dulbecco's Modified Eagle's Medium (DMEM), DMEM/F-15, Liebovitz L-15, RPMI 1640, Iscove's modified Dubelcco's media (IMDM), Ham's F12 medium, OPTI-MEM SFM (Invitrogen Inc.), or a combination thereof. In particular embodiments, the medium does not include either feeder cells or serum, i.e., the medium is feeder cell-free and serum-free. In particular embodiments, the base medium is composed of 75% Iscove's Modified Dulbecco's Medium, 25% Ham's F12 medium, N-2 and B-27 Supplements (without Vitamin A), bovine serum albumin (BSA), 1-thioglycerol and ascorbic acid. Cell cultures may be maintained in a CO₂ atmosphere, e.g., 5% to 12%, to maintain pH of the culture fluid, incubated at 37° C. in a humid atmosphere and passaged to maintain a confluence below, e.g., 85%.

In accordance with the method herein, the base medium is supplemented with BMP4 (e.g., 2 ng/ml human BMP4 commercially available from R&D Systems), VEGF (e.g., 50 ng/ml human VEGF¹⁶⁵ commercially available from Miltenyi Biotec), and basic FGF (e.g., 10 ng/ml human basic FGF commercially available from Millipore). The type IV collagen (GENBANK Accession Nos. NP_034062 and NP_034061) can be obtained from any suitable source and is preferably coated on the surface of the culture dish, plate or container. By way of illustration, type IV collagen can be purified from Engelbreth-Holm Swarm lathrytic mouse tumor, composed exclusive of α1(IV) and α2(IV) chains (Wisdom, et al. (1992) Connect Tissue Res. 27:225-34), and coated on the surface of a 35 or 60 mm dish at a final concentration of 1 to 10 μg/cm² of growth surface.

The step of culturing iPS or ES cells can be carried out for at least, about or up to 1, 2, 3, 4, 5, 6, 7, 8, 9, or 10 days (or any range derivable therein) to induce differentiation into endothelial cells. In some embodiments, iPS or ES cells are cultured for between about 1 to about 5 days. In other embodiments, iPS or ES cells are cultured for about 5 days.

After the cells are cultured, differentiated cells are isolated to obtain an enriched population of cells expressing Flk1 and VE-cadherin, i.e., the endothelial cells of interest. Isolation of the endothelial cells can be carried out by dissociating or dispersing the cultured cells, e.g., by mechanical or enzymatic means (e.g., as trypsin or trypLE, or a mixture of enzymes such as ACCUTASE) into essentially individual cells and isolating the endothelial cells based upon one or more endothelial cell characteristics. For example, endothelial cell can be isolated based upon expression of one or more endothelial cell markers, one or more functional characteristics of endothelial cells, or one or more morphological characteristics of endothelial cells.

In some embodiments, the endothelial cells are isolated by fluorescence-activated cell sorting (FACS). In particular embodiment, endothelial cells are isolated by FACS using anti-Flk1 and anti-VE-cadherin antibodies. As is known in the art, FACS separates a population of cells into sub-populations based on fluorescent labeling. In particular, cells stained using fluorophore-conjugated antibodies can be separated from one another depending on which fluorophore they have been stained with. In accordance with certain embodiments of the present method, a combination of fluorophore-conjugated anti-Flk1 and anti-VE-cadherin antibodies or fluorophore-conjugated secondary antibodies are used to isolate endothelial cells that express both Flk1 and VE-cadherin. In accordance with other embodiments of the present method, an enriched population of cells expressing Flk1, VE-cadherin, and CD31 are isolated. Such cells can be isolated by FACs using a combination of anti-Flk1, anti-VE-cadherin and anti-CD31 antibodies.

In addition to positive selection, i.e., isolation based upon the expression of Flk1, VE-cadherin, and optionally CD31, endothelial cells can also be isolated based upon negative selection. In particular, cells expressing one or both of CD14 and CD45 are preferably excluded from the population of endothelial cells prepared by the method of this invention, i.e., the endothelial cells are negative for CD14, CD45, or a combination thereof. CD14⁺ and/or CD45⁺ cells can be sorted from cells expressing Flk1, VE-cadherin, and optionally CD31 by FACs using an anti-CD14 or anti-CD45 antibody.

Any suitable fluorophore and antibody can be used in the method of this invention. Exemplary fluorphores include, but are not limited to, ALEXA FLUOR dyes (e.g., 488, 546, 610, 700, 647, and 750), R-phycoerythrin, allophycocyanin, TEXAS RED, PE CY5, fluorescein, and QDOT nanocrystals (e.g. 655, 706 and 800). Antibodies suitable for use in flow cytometry are known in the art and readily available from commercial sources such as eBioscience, Fitzgerald Industries International, Biogems International, Abbiotec, and R&D Systems.

In certain embodiments, the step of isolating the endothelial cells can be repeated one, two, three, four, five or more times to obtain a population of endothelial cells that are substantially free of any undesired cell types. Cells are “substantially free” of certain undesired cell types when they have less that 10% of the undesired cell types. However, even more desirable are cell populations wherein less than 0.5% or less than 0.1% of the total cell population is the undesired cell types. Thus, cell populations wherein less than 0.1% to 1% (including all intermediate percentages) of the cells of the population include undesirable cell types are deemed a “substantially pure population of endothelial cells” in accordance with this invention.

Once isolated, the cells can be characterized according to a number of phenotypic criteria. The criteria include but are not limited to the detection or quantitation of expressed cell markers, enzymatic activity, and the characterization of morphological features and intercellular signaling.

In particular embodiments, endothelial cells generated in accordance with this invention express or co-express (i.e., simultaneously express) Flk1 and VE-cadherin. In other embodiments, endothelial cells may express one or more other endothelial cell markers including, but are not limited to, ACE (angiotensin-converting enzyme; (CD143), BNH9/BNF13, CD31, CD34, CD54 (ICAM-1), CD62E (E-Selectin), CD105 (Endoglin), CD146, Endocan (also called ESM-1), Endoglyx-1, Endomucin, Eotaxin-3, EPAS1 (Endothelial PAS domain protein 1), Factor VIII related antigen, FLI-1, FLT-1 (VEGFR-1), GATA2, GBP-1 (guanylate-binding protein-1), GRO-alpha, HEX, ICAM-2 (intercellular adhesion molecule 2), LMO2, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), Thrombomodulin (TM, CD141), VCAM-1 (vascular cell adhesion molecule-1) (CD106), VEGF (Vascular endothelial growth factor), vWF (von Willebrand factor, also called Factor VIII), ZO-1, endothelial cell-selective adhesion molecule (ESAM), CD102, CD93, CD184, CD304, and DLL4. In particular embodiments, the endothelial cells further express CD31. In some embodiments, the endothelial cells produced by the method of this invention do not express certain markers or exhibit decreased expression of certain markers, such as markers of mesenchymal cells (e.g., Brachyury), markers of hematopoietic cells (e.g., CD45 and CD14) or markers of human pluripotent stem cells (e.g., c-Myc, Klf4, Oct4, and Sox2).

Tissue-specific (e.g., endothelial cell-specific) protein and oligosaccharide determinants listed in this disclosure can be detected using any suitable immunological technique including, e.g., flow immunocytochemistry for cell-surface markers, immunohistochemistry (for example, of fixed cells or tissue sections) for intracellular or cell-surface markers, western blot analysis of cellular extracts, and enzyme-linked immunoassay, for cellular extracts or products secreted into the medium. Expression of an antigen by a cell is said to be “antibody-detectable” if a significantly detectable amount of antibody will bind to the antigen in a standard immunocytochemistry or flow cytometry assay, optionally after fixation of the cells, and optionally using a labeled secondary antibody or other conjugate (such as a biotin-avidin conjugate) to amplify labeling.

The expression of tissue-specific (e.g., endothelial cell-specific) markers can also be detected at the mRNA level by northern blot analysis, dot-blot hybridization analysis, or by real-time polymerase chain reaction (RT-PCR) using sequence-specific primers in standard amplification methods (U.S. Pat. No. 5,843,780). Sequence data for the particular markers listed in this disclosure can be obtained from public databases such as GENBANK. Expression at the mRNA level is said to be “detectable” according to one of the assays described in this disclosure if the performance of the assay on cell samples according to standard procedures in a typical controlled experiment results in clearly discernable hybridization or amplification product within a standard time window. Unless otherwise required, expression of a particular marker is indicated if the corresponding mRNA is detectable by RT-PCR. Expression of tissue-specific markers as detected at the protein or mRNA level is considered positive if the level is at least 2-fold, and preferably more than 10- or 50-fold above that of a control cell, such as an undifferentiated ES cell, iPS cell, a fibroblast, or other unrelated cell type.

Cells can also be characterized according to whether they display enzymatic activity that is characteristic of cells of the endothelial lineage. For example, assays that detect uptake of acetylated low density lipoprotein, bradykinin degradation, angiotensin I conversion, or nitric oxide production may be useful. See, e.g., Voyta, et al. (1984) J. Cell Biol. 99:2034-2040; King, et al. (1989) Am. J. Physiol. Cell Physiol. 256:C1231-C1238; Graf, et al. (1992) J. Cardiovas. Pharm. 20(Suppl. 9):516-820; Ming, et al. (2002) Molec. Cell. Biol. 22(24):8467-8477.

In certain embodiments, endothelial cells of this invention have morphological features characteristic of endothelial cells in nature. The features are readily appreciated by those skilled in evaluating such things, and include a squamous appearance and a large central nucleus. One or more such features present in a single cell are consistent with the cell being a member of the endothelial cell lineage. Unbiased determination of whether cells have morphologic features characteristic of endothelial cells can be made by blind analysis of micrographs of the cells of interest, adult or fetal endothelial cells, and one or more negative control cells, such as a fibroblast, or RPE (Retinal pigment epithelial) cells. In other embodiments, cells of the invention are assayed for the ability to form tube-like structures in a three dimensional matrix, such as MATRIGEL, or to respond to pro-inflammatory stimuli (e.g., TNF and/or IL-1) by upregulating the expression of one or more cell-adhesion molecules such (e.g., CD54 (ICAM-1) and/or CD62E). See, e.g., Chalupowicz, et al. (1995) J. Cell Biol. 130(1):207-215.

Endothelial cells provided by this invention can have a number of the features of endogenous endothelial cells they are intended to represent. The more of these features that are present in a particular cell, the more it can be characterized as a cell of the endothelial cell lineage. Cells having at least 2, 3, 5, 7, or 9 of these features are preferred. In reference to a particular cell population as may be present in a culture vessel or a preparation for administration, uniformity between cells in the expression of these features is often advantageous. In this circumstance, populations in which at least about 40%, 60%, 80%, 90%, 95%, or 98% of the cells have the desired features are preferred.

As is known in the art, endothelial cells form the inner lining of a blood vessel and provide an anticoagulant barrier between the vessel wall and blood. In addition to forming a selective permeability barrier, endothelial cells are unique multifunctional cells with critical basal and inducible metabolic and synthetic functions. The endothelial cell reacts with physical and chemical stimuli within the circulation and regulates hemostasis, vasomotor tone, and immune and inflammatory responses. In addition, the endothelial cell is pivotal in angiogenesis and vasculogenesis. Indeed, as demonstrated herein, endothelial cells prepared according to the method of the invention are capable of further differentiating into vessels in vivo.

Accordingly, endothelial cells produced by the method of this invention have a number of uses. These include, but are not limited to, transplantation or implantation of the endothelial cells in vivo; screening cytotoxic compounds, carcinogens, mutagens growth/regulatory factors, pharmaceutical compounds, etc., in vitro; elucidating the mechanism of cardiovascular diseases and injuries; studying the mechanism by which drugs and/or growth factors operate; diagnosing and monitoring cancer in a patient; gene therapy; and the production of biologically active products, e.g., agents to target and treat tumors.

In particular, the endothelial cells of the invention are used to promote vascularization or repair of injured or ischemic tissue in a subject. More specifically, endothelial cells prepared by method disclosed here can be used in a method for treating tissue injury associated with vascular diseases, cardiovascular diseases, ischemic diseases, or other tissue injury (such as, e.g., by engineering of grafts), or hypertension, as disclosed in, for example, Dzau, et al. (2005) Hypertension 46:7-18 and Li, et al. (2009) J. Cell Biochem. 106:194-199. Thus, the endothelial cells of this invention can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Moreover, these cells are of use in preventing critical limb ischemia (CLI) thereby eliminating the need for limb amputation; inducing angiogenesis and myogenesis in patients undergoing cardiac bypass surgeries; screening drugs and validation, e.g., anti-vascular drugs; delivery of drugs/toxins, e.g., to atherosclerotic and psoriasis lesions; modeling vascular diseases and understanding of birth disorders; used as a vehicle to deliver anti-cancer drugs into growing solid tumors so that metastasis and tumor growth can be prevented; wound healing in diabetic ulcers; and reconstructive tissue engineering and 3D tissue and organ printing.

“Treating” a subject having a disease or disorder means accomplishing one or more of the following: (a) reducing the severity of the disease; (b) arresting the development of the disease or disorder; (c) inhibiting worsening of the disease or disorder; (d) limiting or preventing recurrence of the disease or disorder in patients that have previously had the disease or disorder; (e) causing regression of the disease or disorder; (f) improving or eliminating the symptoms of the disease or disorder; and (g) improving survival.

In specific embodiments, this disclosure provides a method of vascularization or repair of tissue in need of vascular cells or vascularization. This method involves contacting injured or ischemic tissue with a composition containing the isolated endothelial cells prepared by the method herein to promote vascularization or repair in such tissue. In particular embodiments, the isolated endothelial cells incorporate into newly formed CD31+ vascular structures.

The tissue in need of vascular cells or vascularization can be cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

Promoting angiogenesis in a tissue can be beneficial to individuals who have or are at risk to develop a condition including an ischemic condition, e.g., myocardial infarction, congestive heart failure, and peripheral vascular obstructive disease, stroke, reperfusion injury, limb ischemia; neuropathy (e.g., peripheral neuropathy, or diabetic neuropathy), organ failure (e.g., liver failure, kidney failure, and the like), diabetes, rheumatoid arthritis, and osteoporosis.

For therapeutic applications, the endothelial cells of this invention can be provided in a composition containing the endothelial cells and one or more pharmaceutically acceptable carriers, diluents, adjuvants or vehicles. The cells can be administered orally, subcutaneously or parenterally including intravenous, intraarterial, intramuscular, intraperitoneally, and intranasal administration as well as intrathecal and infusion techniques. Implants of the cells are also useful. The subject being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the endothelial cells of the invention.

When administering the cells of the invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for endothelial cell compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells. Sterile injectable solutions can be prepared by incorporating the cells utilized in practicing the present invention in the required amount of the appropriate solvent with various of the other ingredients, as desired.

Pharmaceutical compositions of this invention can also include or be administered in parallel with other agents known for use in promoting angiogenesis and/or vasculogenesis. Such agents include, but are not limited to, acidic or basic fibroblast growth factor; transforming growth factor alpha (TGF-α); tumor necrosis factor (TNF); platelet-derived growth factor (PDGF); vascular endothelial cell growth factor (VEGF); angiogenin; a nicotine receptor agonist (U.S. Pat. No. 8,188,043); a TGF-β blocking agent (see, U.S. Pat. No. 8,754,056); a NO level-enhancing agent such as L-arginine, L-lysine, and peptides enriched with these amino acids which can serve as substrates for NO; an agent that preserves NO activity such as an antioxidant (e.g., tocopherol, ascorbic acid, ubiquinone) or an antioxidant enzyme (e.g., superoxide dismutase); an agent that can enhance NO synthase activity (e.g., tetrahydrobiopterin, or precursors for tetrahydrobiopterin (e.g., sepiapterin)); an prostacyclin level-enhancing agent such as a precursor for prostacyclin, e.g., eicosopentanoic acid or docosohexanoic acid; or a prostanoid such as prostaglandin E1 and its analogues. In certain embodiments, the composition can also include components that facilitate engraftment.

The present endothelial cells or a composition containing such cells can be administered in a manner that results in delivery or migration to or near the tissue in need of repair or vascularization. In some embodiments, the cells are systemically administered and circulate to the tissue in need thereof; or alternatively, locally administered, e.g., delivered directly (by injection, implantation or any suitable means) into the tissue or nearby tissue which is in need of these cells. In other embodiments, the cells are integrated into an artificially engineered tissue prior to implantation.

To determine the suitability of endothelial cells provided herein for therapeutic applications, endothelial cells provided by the method of the invention may be tested in various animal models for their ability to treat cardiovascular diseases, vascular disease, vascular injuries, tissue injuries, and the like. Animal models that may find use in certain aspects of the present invention are discussed in, for example, Dzau, et al. (2005) Hypertension 46:7-18 and Li, et al. (2009) J. Cell Biochem. 106:194-199.

Endothelial cells provided in certain aspects of this invention that demonstrate desirable functional characteristics according to their profile of enzymes, or efficacy in animal models, may also be suitable for direct administration to human subjects in need thereof. For purposes of hemostasis, the cells can be administered at any site that has adequate access to the circulation. Endothelial cells may also be delivered at a site of injury or disease. For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells. The ultimate responsibility for determining the mode of treatment and the appropriate dose lies with the managing clinician.

Certain aspects of the invention include endothelial cells provided herein that form part of a bioengineered tissue graft. Such a tissue graft may be a heart tissue graft (see, e.g., US 2008/0199843), a vascularized tissue graft (see, e.g., US 2007/0299508), or any other tissue graft known in the art (see, e.g., US 2008/0063627; US 2007/0184122; US 2007/0141037; US 2010/0145444; US 2009/0324683; US 2009/0149569; US 2007/0122388).

The foregoing may be better understood by reference to the following Examples, which are presented solely for purposes of illustration and are not intended to limit the scope of the invention.

### EXAMPLE1

### Materials and Methods

Reagents. Mouse induced Pluripotent Stem (iPS) cells (iMZ-9 and iMZ-21) are known in the art (Boland, et al. (2009) Nature 461:91-94; Quinlan, et al. (2011) Cell Stem Cell 9:366-373). Undifferentiated mouse embryonic stem cells (mES cells, J1 line) were purchased from American Type Culture Collection (ATCC, Manassas, VA). Rat anti-mouse CD31 antibody (IgG₂ₐ) was purchased from BD Biosciences/Pharmingen (San Jose, Calif.). Anti-Spl antibody was bought from Abcam (Cambridge, Mass.). Anti-Etv2/Er71 (W-14) was purchased from Santa Cruz (Santa Cruz, Calif.). Goat anti-Rat IgG2α FITC was purchased from Bethyl Laboratories (Montgomery, Tex.).

Cell Culture. The mouse ESC line (J1), and iPS iMZ-9 and iMZ-21 cells (Boland, et al. (2009) Nature 461:91-94; Quinlan, et al. (2011) Cell Stem Cell 9:366-373) were propagated and maintained using mitomycin-blocked mouse embryonic fibroblast cells (MEF) and Leukemia Inhibitory Factor (LIF; Chemicon/Millipore, Billerica, Md.). Briefly, mESCs or iPSCs were cultured in the ES cell medium composed of high glucose-Dulbecco's Modified Eagles medium (DMEM; Invitrogen, Carlsbad, Calif.) supplemented with 15% ES-qualified fetal bovine serum (FBS; Invitrogen), 2 mM L-glutamine (Invitrogen), 1 mM sodium pyruvate, 0.1 mM nonessential amino acids (Invitrogen), penicillin (10 μg/ml), streptomycin (5 μg/ml), 0.1 mM β-mercaptoethanol (Invitrogen), and 500 U/ml recombinant LIF (ESGRO®; Millipore) at 37° C. with 5% CO₂. The cells were passed every 3 to 4 days at a ratio of 1:15 using a 1 mM EDTA phosphate-buffered saline (PBS) solution as the dissociation buffer. For the differentiation studies, mESC or iPSC cells were passed twice in gelatin-coated culture dishes at a 1:3 dilution without the presence of MEF feeders. To initiate differentiation, the MEF-free pre-conditioned cells were dissociated with 1 mM EDTA PBS and seeded on type IV collagen-coated dishes (BD Bioscience, San Jose, Calif.) at a density of 35,000/35 nun dishes in serum-free differentiation medium composed of 75% Iscove's Modified Dulbecco's Medium (IMDM), 25% Ham's F12 medium, N-2 and B-Supplements (without Vitamin A), 0.05% BSA (all from Gibco), 4.5×10e-4 M 1-thioglycerol (MTG) and 0.5 mM ascorbic acid (both from Sigma, St Louis, Mo.). The serum-free medium was supplemented with human BMP-4 at 2 ng/ml (R&D Systems), human VEGF¹⁶⁵ at 50 ng/ml (Miltenyi Biotec), and human basic FGF at 10 ng/ml (Millipore).

Flow Cytometric Analysis and Sorting for Subcultures. Differentiated mES and iPS cells were collected following dissociation with a 1 mM EDTA PBS solution. Single-cell suspensions were prepared and labeled with goat-anti-mouse VE-cadherin (R&D Systems, Minneapolis, Minn.) and donkey anti-goat secondary antibody coupled with ALEXA FLUOR 488 dye (AF-488) (eBioscience, San Diego, Calif.). The other primary antibodies used for the analysis or sorting included rat anti-mouse Flk1(clone avas 12a) coupled with allophycocyanin (APC) (eBioscience) and rat-anti-mouse CD41 coupled with R-phycoerythrin (BD Bioscience). Immunofluorescence-labeled cell populations bearing specific marker(s) were analyzed on a CYAN ADP Analyzer (Beckman Coulter) or sorted using a MOFLO high speed sorter (Becton Dickinson, Franklin Lakes, N.J.). To test the lineage characteristics and colony formation of the sorted cells, the cells were subcultured at 50,000 per 35 mm type IV collagen BIOCOAT dishes with the serum-free differentiation medium. The number and identities of endothelial and hematopoietic colonies were counted and confirmed in situ using goat anti-mouse VE-cadherin (R&D Systems, Minneapolis, Minn.) and donkey anti-goat secondary antibody coupled with AF-488 (eBioscience) as well as rat-anti-mouse CD41 coupled with R-phycoerythrin (BD Biosciences) for the early hematopoietic lineages.

Gene Expression Analysis. The profile of pluripotent, mesoderm, hemangioblast, angioblast, hematopoietic and mature EC markers were quantified using quantitative (q) RT-PCR as previously described (Kohler, et al. (2011) Blood 117:1761-1769; Wary, et al. (2009) Stem Cells 27:3112-3120). Q-RT-PCR assays were performed using the ABI PRISM 7700 Sequence Detection System (Applied Biosystems, Carlsbad, Calif.) according to the manufacturer's instructions. Oligonucleotides used in this analysis are presented in Table 1. The experiments were carried out 5 times at least in triplicate for each gene target.

TABLE 1

| | | | SEQ ID | Accession |
|---|---|---|---|---|
| Gene | Primer | Sequence | NO: | No. |
| c-Myc | Forward | ACAAGCTCACCT | 1 | L00038 |
| | | CTGAAAAGGACT | | |
| | Reverse | CTCGAGTTAGGT | 2 | |
| | | CAGTTTATGCAC | | |
| Oct4 | Forward | CCACTTCACCAC | 3 | NM_013633 |
| | | ACTCTACTCAG | | |
| | Reverse | AAGCTCCAGGTT | 4 | |
| | | CTCTTGTCTAC | | |
| Gapdh | Forward | GACAATGAATAC | 5 | GU214026 |
| | | GGCTACAGCA | | |
| | Reverse | GTTATTATGGGG | 6 | |
| | | GTCTGGGATG | | |
| Klf4 | Forward | GAGGAAGCGATT | 7 | NM_010637 |
| | | CAGGTACAGAAC | | |
| | Reverse | AGGCTTATTTAC | 8 | |
| | | CTGGCTTAGGTC | | |
| Sox2 | Forward | CTAGTGGTACGT | 9 | U31967 |
| | | TAGGCGCTTC | | |
| | Reverse | GCCCGGAGTCTA | 10 | |
| | | GCTCTAAATA | | |
| Brachyury | Forward | CATTACACACCA | 11 | BC120807 |
| | | CTGACACACAC | | |
| | Reverse | AGTCTCAGCACA | 12 | |
| | | TGGAGGAAAGT | | |
| Er71 | Forward | GACTACACCACC | 13 | L10427 |
| | | ACGTGGAATAC | | |
| | Reverse | AGACTGCTTGTT | 14 | |
| | | CGATTTGGAG | | |
| Flk1 | Forward | TGTGGTCTCACT | 15 | NM_010612 |
| | | ACCAGTTAAAGC | | |
| | Reverse | CATTCGATCCAG | 16 | |
| | | TTTCACAGAG | | |
| VE | Forward | AGATCCCAGAAG | 17 | NM_009868 |
| cadherin | | AGCTAAGAGGAC | | |
| | Reverse | AGAAAAGGAAGA | 18 | |
| | | GTGAGTGACCAG | | |
| CD31 | Forward | GAGACTCAGAGG | 19 | NM_008816 |
| | | CGCTAGTTAAT | | |
| | Reverse | CTAACCCAGTGA | 20 | |
| | | TTGACAACAGA | | |

Chromatin Immunoprecipitation (ChIP) Assay. ChIP methodologies are known in the art (Kohler, et al. (2011) Blood 117:1761-1769; Cowan, et al. (2010) Circ. Res. 107:959-966). ChIP kit was purchased from Thermo Fisher Scientific (Rockford, Ill.). The chromatins were pre-cleared and then subjected to immunoprecipitation with specific antibodies. Anti-Glut-1 and anti-Sp-1 were used as controls. The immunoprecipitated DNAs were analyzed by PCR using primer pairs that amplify the region of the Flk1-, VE-cadherin-, and CD31-promoter/enhancer DNA sequences.

MATRIGEL Assay, Hind Limb Ischemia, Immunofluorescence and Microscopy. Three-month-old athymic nude mice (Harlan Laboratory, Madison, Wis.) were used for this assay. All experiments involving nude and C57 mice reported in this study were conducted according to Institutional Animal Care and Use Committee and NIH guidelines. The mice were housed in the University of Illinois at Chicago Animal Care Vivarium under pathogen-free conditions and treated according to the UIC protocol for Animal Care Committee (ACC). For post-surgical pain management buprenex was used. The mouse ES cell line (J1) and iPS (iMZ21) cell line derived Flk1⁺/VE-cadherin⁺ cells were incubated in lentivirus encoding mCherry (BioVision, Mountain View, Calif.) in media containing mCherry (˜10⁷ particles/ml) overnight in complete differentiation media (Kohler, et al. (2011) Blood 117:1761-1769; Wary, et al. (2009) Stem Cells 27:3112-3120). Growth factor-reduced 200 μL MATRIGEL (BD Biosciences, San Jose, Calif.) +2 million mCherry-treated cells +30 μl VEGF¹⁶⁵ (Lonza; Walkersville, Md.) +2 ng/μl Wnt3a (R&D Systems) were injected subcutaneously into the midventral abdomen of the mice. Plugs were allowed to solidify, and the mice were monitored every 24 hours to assess the wound. After 7 days, the plugs were retrieved, washed in PBS, and fixed with 4% paraformaldehyde. Five-micrometer serial sections were prepared and stained with hematoxylin and eosin (H&E). Femoral artery ligation to induce hind limb ischemia in C57 mice is described in the art (Urao, et al. (2008) Circ. Res. 103:212-220). Immunofluorescent staining, microscopy, and quantification have been described in the art (Kohler, et al. (2011) Blood 117:1761-1769; Wary, et al. (2009) Stem Cells 27:3112-3120; Cowan, et al. (2010) Circ. Res. 107:959-966). For quantification, 10-12 microscopic fields were randomly selected, with 10× or 20× magnifications in six different sections. A digital camera was used to capture images, which were then saved as TIFF documents. Composite figures were assembled and labeled with the use of QuarkXpress 8.1.2 software (Quark Inc., Denver, Colo.), and then the images were converted into the EPS document.

Statistical Analysis. GraphPad Prizm 5.0 (GraphPad Software, Inc., La Jolla, Calif.) was used to analyze data as (Cowan, et al. (2010) Circ. Res. 107:959-966; Urao, et al. (2008) Circ. Res. 103:212-220). The data represent mean±S.E.M. Analysis of variance (ANOVA) was performed with posthoc comparisons using an unpaired t test or Mann-Whitney tests, as appropriate. P<0.05 is considered significant.

### EXAMPLE 2

### Differentiation of mES and miPS Cells Into Flk1⁺VE-Cadherin⁺ Cells

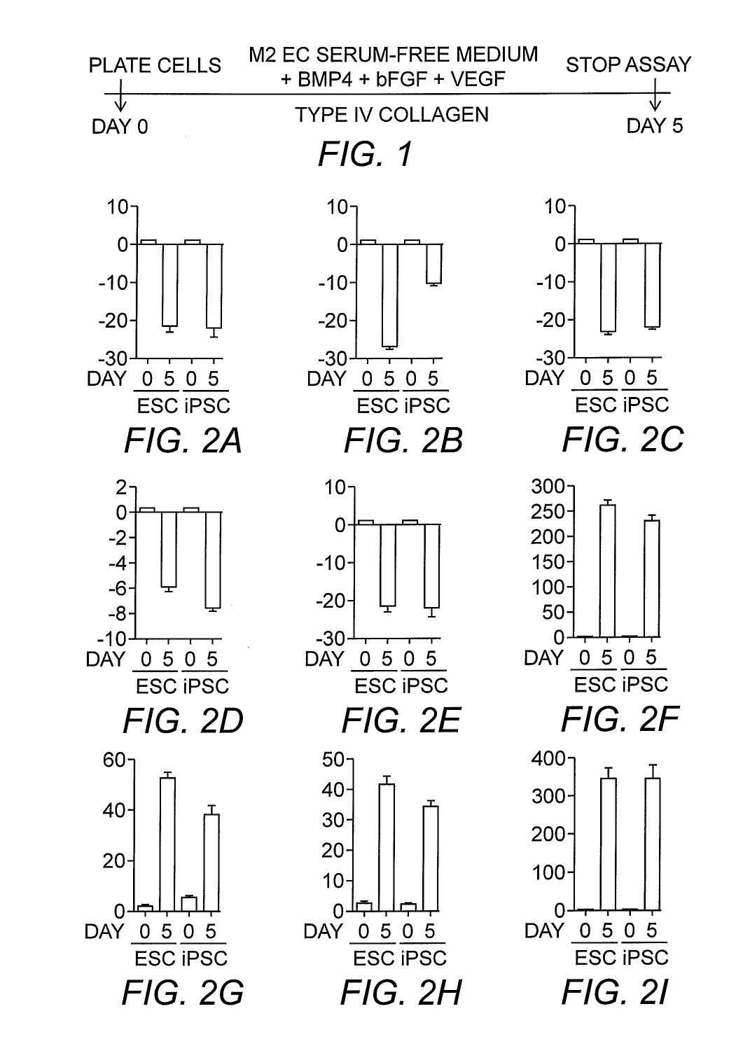

Since mesodermal-ECs are anchorage-dependent cells known to adhere onto type IV collagen, type IV collagen was used as the supporting matrix protein in these studies. The experimental timeline and differentiation conditions are as shown (FIG. 1). To induce formation of Flk1⁺ vascular progenies, media containing LIF was removed from naïve mES and miPS cells, and the cells were cultured on type IV collagen-coated dishes and propagated in presence of complete media containing BMP4, VEGF, and bFGF. Using mouse ES (J1 line) and iPS (iMZ-21 line) cells in this culture condition, the emergence of adherent cells was observed on day 5. The appearance of Flk1⁺ or VE-cadherin⁺ cells was monitored using anti-mouse-Flk1 and anti-mouse-VE-cadherin monoclonal antibodies and the cells were sorted using fluorescence-activated cell sorting (FACS). mES cells gave rise to 38±5% Flk1⁺ cells on day 2 and peaked at 49±7% on day 3, whereas these numbers dropped to 46±5% and 37±6% on days 4 and 5, respectively. The yield of VE-cadherin⁺ cells from mES was 7±3% on day 2 and increased steadily to 23±3%, 47±5% and 52±4% on days 3, 4 and 5, respectively. The emergence of Flk1⁺ and VE-cadherin⁺ cells from miPS cells followed similar trends. Percentage of Flk1⁺ cells derived from iPS cells were 32±3%, 48±4%, 42±3%, 35±3% on days 2, 3, 4 and 5, respectively; while the yield of VE-cadherin⁺ cells from miPS was 6±3% on day 2 and increased steadily to 25±3%, 32±3% and 37±2% on days 3, 4 and 5, respectively. The co-expression of Flk1 and VE-cadherin was used as an indicator of true vascular differentiation. Hematopoietic clusters made of round cells were not quantified.

After establishing day 5 as the optimal time for generation of Flk1⁺ VE-cadherin⁺ cells from both miPS and mES cells, this time point was used for all subsequent studies. Day 5 cells were subjected to a two-step purification procedure using anti-Flk1 and anti-VE-cadherin antibodies to sort Flk1 and VE-cadherin double-positive cells. In the first purification step, sorting of mES cells gave rise to 40±9% Flk1⁺ and VE-cadherin⁺ cells, whereas miPS cells yielded 35±10% Flk1⁺ and VE-cadherin⁺ cells. Upon subjecting these cells to a second round of purification, nearly 100% Flk1⁺ and VE-cadherin⁺ cells were obtained. Further analysis of the gated Flk1+ and VE-cadherin⁺ cells derived from mES and miPS cells showed that they lacked CD14 and CD45. In addition, anti-VE-cadherin and anti-CD31 antibodies staining confirmed the EC nature of the day 5 Flk1⁺VE-cadherin⁺cells derived from mES cells as well as miPS cells.

### EXAMPLE 3

### mRNA Expression Profile of mES and miPS Cell-Derived ECs

mRNAs were prepared and the expression of pluripotent stem cell markers c-Myc, Klf4, Oct4, and Sox2; the mesodermal/mesenchymal markers Brachyury and Er71; and mature EC markers Flk1, VE-cadherin, and CD31 was assessed by q-RT-PCR on days 0 and 5. Naive (undifferentiated) mES and iPS cells expressed c-Myc, Klf4, Oct4, Sox2 on day 0, whereas expression decreased significantly by day 5 (FIGS. 2A-2D). Because Brachyury and Er71 are critical for mesodermal/EC fate (Choi, et al. (1998) Development 125:725-732; Choi, et al. (1998) Development 125:725-732; Huber, et al. (2004) Nature 432:625-630; Kataoka, et al. (2011) Blood 118:6975-6986; Palencia-Desai, et al. (2011) Development 138:4721-4732; Liu, et al. (2012) Blood 119:3295-305), their expression was examined in mES-and miPS-cells and in ECs derived from mES-and miPS-cells (FIGS. 2E and 2F). Expression of the mesodermal marker Brachyury decreased on day 5 cells but Er71 expression remained high on day 5 in cells obtained from both mES and miPS cells (FIG. 2F). Importantly, the expression of the EC markers Flk1, VE-cadherin and CD31 in both mES and miPS cells was low on day 0, but increased significantly by day 5 (FIG. 2G-2I). However, expression of the control Gapdh remained unaltered in all cells. Expression of the hematopoietic cell markers CD45 and CD14 genes was low throughout.

### EXAMPLE 4

### Er71 Binds to Flk1-, VE-cadherin-, and CD31-Promoters During Differentiation to Endothelial Lineage

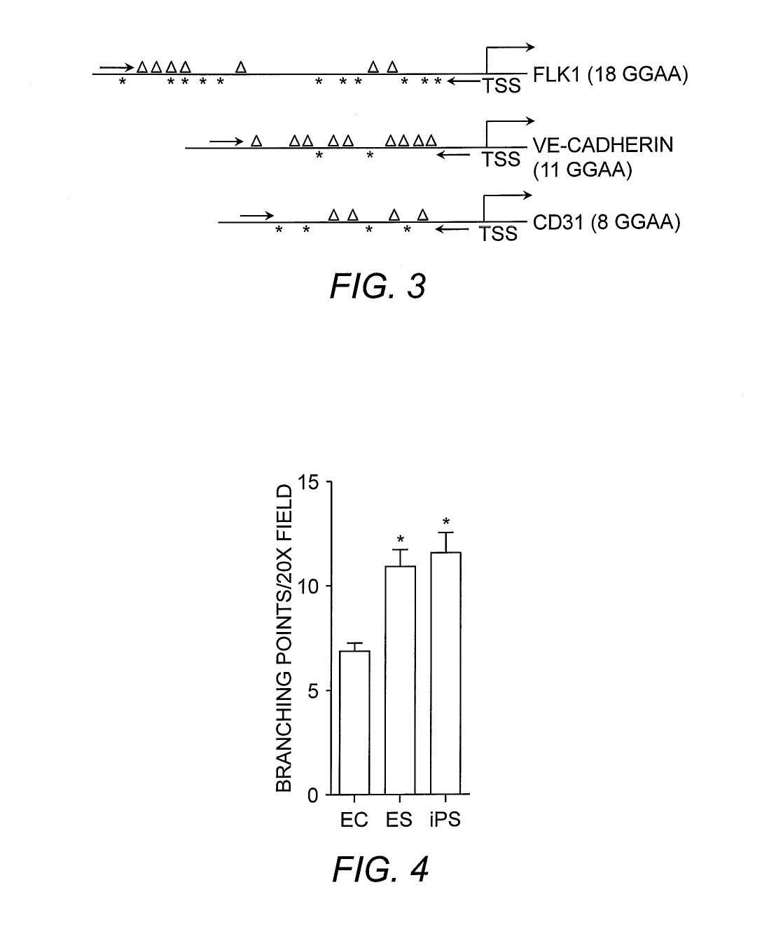

Expression of the Ets transcription factor Er71 was elevated in cells obtained from both mES and miPS cells (FIG. 2F). Because Er71 regulates Flk1 and VE-cadherin expression in physiological vascular development (Park, et al. (2013) Circ. Res. 112:1380-400; Lee, et al. (2008) Cell Stem Cell 2:497-507; Kataoka, et al. (2011) Blood 118:6975-6986; Rasmussen, et al. (2011) Development 138:4801-4812), it was determined whether Er71 was functional in Flk1⁺ and VE-cadherin⁺ ECs derived from mES-and miPS-cells. DNA sequences identified putative Er71 binding sites (GGAA) within the −0.7 kb to −1.4 kb upstream genomic segment of transcription starts sites (TSS) of Flk1, VE-cadherin, and CD31 promoter DNA sequences (FIG. 3). The expression of Er71 and VE-cadherin proteins was confirmed by western blot analysis. A ChIP assay was used to identify sites bound by Er71 in the promoters of Flk1, CD31, and VE-cadherin of ECs obtained from either mES or iPS cells. Immunoprecipitation studies were carried out using anti-Glut-1 (control), anti-Sp1, and anti-Er71 antibodies. To analyze protein-DNA complex formation, specific PCR primers were designed (Table 2). Using these primers, no binding of Glut-1 or Sp1 to Flk1, VE-cadherin, or CD31 DNA sequences was detected. By contrast, Er71 bound similarly to Flk1, VE-cadherin, and CD31 promoter sequences in ECs obtained from both mES and iPS cells. As Sp-1 constitutively occupies GGAA sites in the genome, anti-Sp1 antibody was included as an additional control for the ChIP experiments. ChIP analysis was shown to be specific for Er71 because Flk1, VE-cadherin, and CD31 promoters were not detected using the anti-Glut-1 and anti-Sp1 antibodies.

TABLE 2

| | | | SEQ ID |
|---|---|---|---|
| Gene | Primer | Sequence (5′ → 3′) | NO: |
| Flk1 | Forward | TGCCAGGAGTAAAACATGTCAC | 21 |
| | Reverse | AGTTCTCAGGCACAGACTCCTT | 22 |
| VE- | Forward | CTCGAACTCAGAAATCCACCTG | 23 |
| cadherin | Reverse | GGGAGTCTCACTTACCTTGTCC | 24 |
| CD31 | Forward | ATACACCCACAGTTCCACACAA | 25 |
| | Reverse | ACCTCAGCTGAATCCTGAAAGAC | 26 |

### EXAMPLE 5

### Flk1⁺VE-Cadherin⁺ ECs Derived From mES- and miPS-Cells Form Vascular Networks

ECs plated onto MATRIGEL in vitro are induced to undergo a morphogenic differentiation into vascular-plexus like networks or branching point structures, a process requiring cell adhesion and growth factor stimulation. To determine the neovascularization potential of the Flk1⁺VE-cadherin⁺ cells, the cells were first seeded onto growth factor-reduced (GFR) MATRIGEL in vitro and formation of branching points was quantified (FIG. 4). Plating of mature Flk1⁺VE-cadherin⁺ ECs (control) onto MATRIGEL-coated dishes resulted in spontaneous assembly of branching point structures within 18 hours. Plating of Flk1⁺VE-cadherin⁺ cells derived from either mES or miPS cells on growth factor-reduced MATRIGEL resulted in similar formation of branching point networks. In the absence of VEGF, however, Flk1⁺VE-cadherin⁺ ECs failed to form interconnecting branching point networks. Flk1⁻VE-cadherin⁻ cells did not form branching point structures. The Flk1⁺VE-cadherin⁺ cells derived from mES and miPS cells both functioned similarly and responded to VEGF to form branching point structures.

### EXAMPLE 6

### Mouse ES or iPS Cell-Derived Flk-1⁺VE-Cadherin⁺ Cells Incorporate into CD31⁺ Vessels in situ

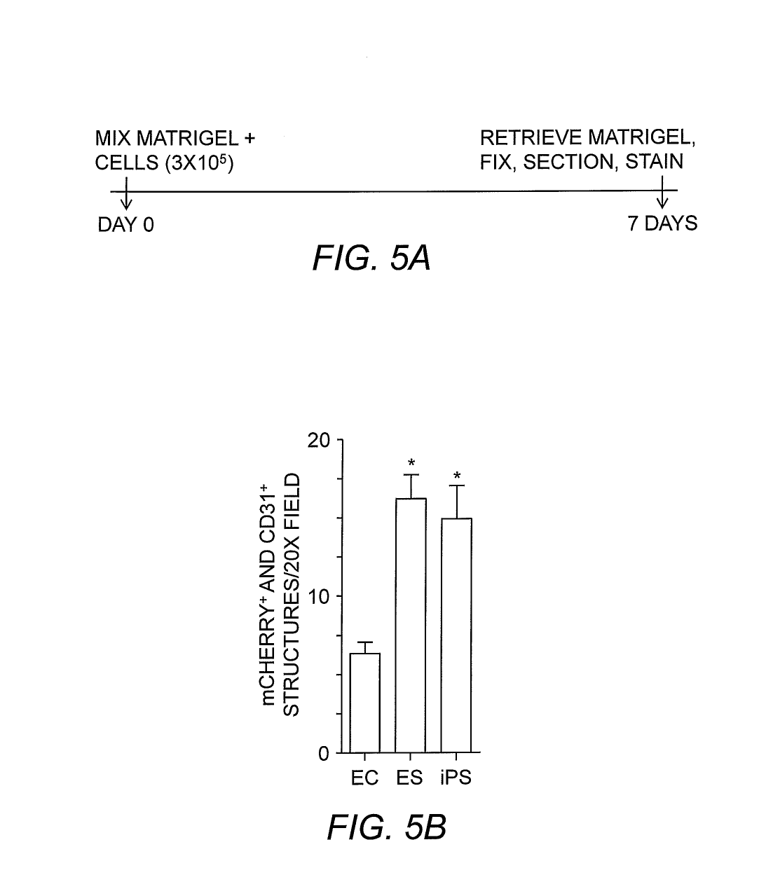

To address whether mES-and miPS-derived Flk-1⁺VE-cadherin⁺ cells incorporated into vessels, a MATRIGEL plug assay was carried out using athymic nude mice. Both mES and miPS cells were differentiated as described above, and Flk1⁺VE-cadherin⁺ cells were sorted by FACS as described herein. The timeline of the experiment is shown in FIG. 5A. Prior to mixing with the MATRIGEL, Flk-1⁺VE-cadherin⁺ cells were incubated overnight with lentiviral particles encoding the mCherry gene. Detachment of cells or cell death after lentivirus-mCherry transfection was not observed. Moreover, the morphology of lentivirus-transfected cells appeared normal, and transfection efficiency of mCherry was 100%. MATRIGEL was prepared and mixed with mCherry-expressing cells, and then injected subcutaneously. MATRIGEL plugs became visibly vascularized within 3-4 days. After 7 days, MATRIGEL plugs were collected and fixed, and formation of vascular structures was analyzed by H&E and anti-mouse CD31 staining. Quantification demonstrated that Flk1⁺VE-cadherin⁺ cells obtained from mES and miPS cells were similar in their ability to incorporate into CD31⁺ neovessels, and did so in higher numbers compared with mature mECs (FIG. 5B). Immunofluorescent histochemistry of MATRIGEL sections with anti-mouse CD31 showed neovessels containing both donor (Flk1⁺VE-cadherin⁺, mCherry) and host cells.

Since ischemia is known to mobilize adult progenitor cells (Takahashi, et al. (1999) Nat. Med. 5:434-438), it was determined whether the Matrigel-engrafted iPSC-ECs would be mobilized by an ischemic insult. Mice were subjected to hindlimb ischemia 7 days after implantation of MATRIGEL containing either no cells, mouse ECs or iPS cell-derived Flk1+VE-cadherin+ ECs. To enable tracking of the cells, ECs were transduced with mCherry lentivirus particles before mixing with MATRIGEL. All animals survived the surgery and appeared to be healthy during the initial post-operative monitoring period. However, an appreciable number of mCherry positive cells in the ischemic TA muscle was not obsevered beyond the autofluorescence signal that was also seen in mice that did not receive any cells. These data collectively show that the MATRIGEL-bound cells did not mobilize to areas of injury, whereas iPS-ECs showed long-term stability ability by incorporating into CD31⁺ vessels.

What is claimed is:

1. A method of preparing endothelial cells that express Ets-related protein 71 (Er71), Fetal liver kinase-1 (Flk1) and Vascular Endothelial (VE)-cadherin comprising (i) culturing induced pluripotent stem cells or embryonic stem cells in the presence of Type IV collagen, Bone Morphogenetic Protein 4 (BMP4), Vascular Endothelial Growth Factor (VEGF) and basic Fibroblast Growth Factor (bFGF); and (ii) isolating endothelial cells that express transcription factor Er71, and vascular endothelial cell surface markers Flk1 and VE-cadherin proteins.

2. The method of claim 1, wherein the type IV collagen comprises α1(IV) and α2(IV) chains.

3. The method of claim 1, wherein the induced pluripotent stem cells or embryonic stem cells are cultured for about 1 to about 5 days.

4. The method of claim 1, wherein induced pluripotent stem cells or embryonic stem cells are cultured for about 5 days.

5. The method of claim 1, wherein step (ii) comprises isolating endothelial cells co-expressing Flk1 and VE-cadherin by fluorescence-activated cell sorting using anti-Flk1 and anti-VE-cadherin antibodies.

6. The method of claim 5, wherein step (ii) is repeated two or more times.

7. The method of claim 1, further comprising isolating endothelial cells co-expressing Flk1, VE-cadherin and CD31.

8. The method of claim 1, further comprising isolating endothelial cells which are negative for CD14, CD45, or a combination thereof.

9. A substantially pure population of endothelial cells prepared by differentiating induced pluripotent stem cells or embryonic stem cells in the presence of Type IV collagen, Bone Morphogenetic Protein 4, Vascular Endothelial Growth Factor and basic Fibroblast Growth Factor, wherein said endothelial cells express Fetal liver kinase-1 and Vascular Endothelial-cadherin.

10. The substantially pure population of endothelial cells of claim 9, wherein said endothelial cells further express CD31.

11. The substantially pure population of endothelial cells of claim 9, wherein said endothelial cells are negative for CD14 and 45.

12. A composition comprising the substantially pure population of endothelial cells of claim 9 and at least one pharmaceutically acceptable carrier or diluents.

13. A bioengineered tissue graft comprising the substantially pure population of endothelial cells of claim 9.

14. A method of promoting vascularization or repair of injured or ischemic tissue comprising contacting injured or ischemic tissue with the composition of claim 12 thereby promoting vascularization or repair of the injured or ischemic tissue.

15. The method of claim 14, wherein the injured tissue comprises ischemic tissue, cardiac tissue, liver tissue, pancreatic tissue, renal tissue, muscle tissue, neural tissue, or bone tissue.


SEQUENCE LISTING (26 sequences)

SEQ ID NO: 1 - DNA, 24 bp, Artificial Sequence
  Synthetic oligonucleotide
acaagctcacctctgaaaaggact

SEQ ID NO: 2 - DNA, 24 bp, Artificial Sequence
  Synthetic oligonucleotide
ctcgagttaggtcagtttatgcac

SEQ ID NO: 3 - DNA, 23 bp, Artificial Sequence
  Synthetic oligonucleotide
ccacttcaccacactctactcag

SEQ ID NO: 4 - DNA, 23 bp, Artificial Sequence
  Synthetic oligonucleotide
aagctccaggttctcttgtctac

SEQ ID NO: 5 - DNA, 22 bp, Artificial Sequence
  Synthetic oligonucleotide
gacaatgaatacggctacagca

SEQ ID NO: 6 - DNA, 22 bp, Artificial Sequence
  Synthetic oligonucleotide
gttattatgggggtctgggatg

SEQ ID NO: 7 - DNA, 24 bp, Artificial Sequence
  Synthetic oligonucleotide
gaggaagcgattcaggtacagaac

SEQ ID NO: 8 - DNA, 24 bp, Artificial Sequence
  Synthetic oligonucleotide
aggcttatttacctggcttaggtc

SEQ ID NO: 9 - DNA, 22 bp, Artificial Sequence
  Synthetic oligonucleotide
ctagtggtacgttaggcgcttc

SEQ ID NO: 10 - DNA, 22 bp, Artificial Sequence
  Synthetic oligonucleotide
gcccggagtctagctctaaata

SEQ ID NO: 11 - DNA, 23 bp, Artificial Sequence
  Synthetic oligonucleotide
cattacacaccactgacacacac

SEQ ID NO: 12 - DNA, 23 bp, Artificial Sequence
  Synthetic oligonucleotide
agtctcagcacatggaggaaagt

SEQ ID NO: 13 - DNA, 23 bp, Artificial Sequence
  Synthetic oligonucleotide
gactacaccaccacgtggaatac

SEQ ID NO: 14 - DNA, 22 bp, Artificial Sequence
  Synthetic oligonucleotide
agactgcttgttcgatttggag

SEQ ID NO: 15 - DNA, 24 bp, Artificial Sequence
  Synthetic oligonucleotide
tgtggtctcactaccagttaaagc

SEQ ID NO: 16 - DNA, 22 bp, Artificial Sequence
  Synthetic oligonucleotide
cattcgatccagtttcacagag

SEQ ID NO: 17 - DNA, 24 bp, Artificial Sequence
  Synthetic oligonucleotide
agatcccagaagagctaagaggac

SEQ ID NO: 18 - DNA, 24 bp, Artificial Sequence
  Synthetic oligonucleotide
agaaaaggaagagtgagtgaccag

SEQ ID NO: 19 - DNA, 23 bp, Artificial Sequence
  Synthetic oligonucleotide
gagactcagaggcgctagttaat

SEQ ID NO: 20 - DNA, 23 bp, Artificial Sequence
  Synthetic oligonucleotide
ctaacccagtgattgacaacaga

SEQ ID NO: 21 - DNA, 22 bp, Artificial Sequence
  Synthetic oligonucleotide
tgccaggagtaaaacatgtcac

SEQ ID NO: 22 - DNA, 22 bp, Artificial Sequence
  Synthetic oligonucleotide
agttctcaggcacagactcctt

SEQ ID NO: 23 - DNA, 22 bp, Artificial Sequence
  Synthetic oligonucleotide
ctcgaactcagaaatccacctg

SEQ ID NO: 24 - DNA, 22 bp, Artificial Sequence
  Synthetic oligonucleotide
gggagtctcacttaccttgtcc

SEQ ID NO: 25 - DNA, 22 bp, Artificial Sequence
  Synthetic oligonucleotide
atacacccacagttccacacaa

SEQ ID NO: 26 - DNA, 23 bp, Artificial Sequence
  Synthetic oligonucleotide
acctcagctgaatcctgaaagac